SEQUENCE LISTING (1 sequence)

SEQ ID NO: 1 - DNA, 52 bp, Homo sapiens
  misc_feature (27)..(27): N represents C or T
ctgaaccagggagctccccgaaggcgngaaccagggttgaattgcactccgc

(19) United States
(12) Patent Application Publication
(10) Pub. No.: US 2014/0314713 A1
(43) Pub. Date: Oct. 23, 2014

(54) METHODS FOR DETERMINING TREATMENT RESPONSE IN PATIENTS INFECTED WITH HCV GENOTYPE 4

(71) Applicant: Tariq-Yacine Asselah, Clichy (FR)

(72) Inventor: Tariq-Yacine Asselah, Clichy (FR)

(73) Assignee: UNIVERSITE PARIS DIDEROT - PARIS VII, Paris Cedex 13 (FR)

(21) Appl. No.: 14/233,652

(22) PCT Filed: Jul. 20, 2012
(86) PCT No.: PCT/EP2012/064250

§ 371 Date: Feb. 27, 2014

Related U.S. Application Data
(60) Provisional application No. 61/509,659, filed on Jul. 20, 2011.

(30) Foreign Application Priority Data
Jul. 20, 2011 (EP) 11305943.0

(51) Int. Cl. C12Q 1/70

(52) U.S. Cl. CPC C12Q 1/70
USPC 424/85.7; 435/5

(57) ABSTRACT

The present invention relates to a method for testing whether a patient infected with HCV genotype 4 could achieve a sustained virological response (SVR) to the combination of interferon-alpha and ribavirin comprising determining the patient's genotype for the single nucleotide polymorphism rs 12979860 wherein the presence of the better response allele C indicates a high chance to achieve a sustained virological response (SVR) and the presence of the allele T indicates a lower chance to achieve a sustained virological response (SVR).

### FIELD OF THE INVENTION

The present invention relates to a method for testing whether a patient infected with HCV genotype 4 could achieve a sustained virological response (SVR) to the combination of interferon-alpha and ribavirin

### BACKGROUND OF THE INVENTION

Hepatitis C virus (HCV) is a major cause of chronic liver disease, with more than 170 million infected individuals worldwide. Genotype 4 (HCV-4) is the most frequent cause of chronic hepatitis C in the Middle-East, North Africa and sub-Saharan Africa. It has recently spread to southern Europe, particularly among intravenous drug users and in immigrants. HCV-4 is mainly found in Egypt, the country with the highest prevalence of HCV worldwide (15%), where HCV-4 represents 90% of all HCV cases.

For patients with HCV-4 infection, the most effective therapy to eradicate the virus consists of a combination of pegylated interferon (PEG-IFN) alpha and ribavirin.

Unfortunately, the rate of sustained virological response (SVR) averages 50% in genotype 1 and 4 infected patients. Because a significant number of patients will fail to respond or will have significant side effects, it is of major interest for both patient care and economic approach to predict non response.

The sequencing of the human genome, together with the development of high-throughput technologies deliver fast, affordable and accurate genomic information, has afforded unique opportunity to predict treatment response. Several independent genome-wide association studies (GWAS) reported single nucleotide polymorphisms (SNPs) near the IL28B (IFN-λ3) locus that displayed association with treatment response, mainly in genotype 1 infected patients (Ge D, Fellay J, Thompson A J et al. Genetic variation in IL28B predicts hepatitis C treatment-induced viral clearance. Nature 2009; 461:399-401; Suppiah V, Moldovan M, Ahlenstiel G et al. IL28B is associated with response to chronic hepatitis C interferon-alpha and ribavirin therapy. Nat Genet 2009; 41:1100-1104. Tanaka Y, Nishida N, Sugiyama M et al. Genome-wide association of IL28B with response to pegylated interferon-alpha and ribavirin therapy for chronic hepatitis C. Nat Genet 2009; 41:1105-1109; Rauch A, Kutalik Z, Descombes P et al. Genetic variation in IL28B is associated with chronic hepatitis C and treatment failure: a genome-wide association study. Gastroenterology. 2010; 138(4):1338-45, 1345.e1-7. Asselah T. Genetic polymorphism and response to treatment in chronic hepatitis C: the future of personalized medicine. J Hepatol. 2010 March; 52(3):452-4.).

Interestingly, the association between IL28B polymorphism and SVR was not confirmed in other cohorts of genotypes 2 and 3 infected patients. In a cohort of 281 patients infected with HCV genotype 3, there was no association of SNP rs12979860 with SVR to PEGIFN/ribavirin therapy (Moghaddam A, Melum E, Reinton N et al. IL28B genetic variation and treatment response in patients with hepatitis C virus genotype 3 infection. Hepatology. 2011; 53(3):746-54.). Also, the association of rs12979860 with an SVR in patients infected with genotype 2/3 HCV was through only in those who did not achieve a rapid virological response (RVR) (Mangia A, Thompson A J, Santoro R et al. An IL28B polymorphism determines treatment response of hepatitis C virus genotype 2 or 3 patients who do not achieve a rapid virologic response. Gastroenterology. 2010; 139(3):821-7, 827.e1.). Furthermore, in 482 Asian HCV-2 patients treated with the standard of care (SOC) (PEG-IFN plus ribavirin), the rs8099917 polymorphism (near the IL28B gene) played no role in achieving SVR with or without RVR (Yu M L, Huang C F, Huang J F et al. Role of interleukin-28B polymorphisms in the treatment of hepatitis C virus genotype 2 infection in Asian patients. Hepatology. 2011; 53(1):7-13.). The majority of studies focused on genotypes 1, 2 and 3. There is few data so far regarding the role of IL28B polymorphism in HCV-4 patients with respect to response to antiviral therapy or to fibrosis progression (Rauch A, Kutalik Z, Descombes P et al. Genetic variation in IL28B is associated with chronic hepatitis C and treatment failure: a genome-wide association study. Gastroenterology. 2010; 138(4):1338-45, 1345.e1-7.). Therefore, the importance to perform genetic studies in HCV-4 infection has been outlined in different articles (Kamal S M. Hepatitis C virus genotype 4 therapy: progress and challenges. Liver Int. 2011; 31 Suppl 1:45-52.), all the more than the SOC will remain PEG-IFN and ribavirin for those patients.

### SUMMARY OF THE INVENTION

The present invention relates to a method for testing whether a patient infected with HCV genotype 4 could achieve a sustained virological response (SVR) to the combination of interferon-alpha and ribavirin comprising determining the patient's genotype for the single nucleotide polymorphism rs12979860 wherein the presence of the better response allele C indicates a high chance to achieve a sustained virological response (SVR), and the presence of the allele T indicates a lower chance to achieve a sustained virological response (SVR).

### DETAILED DESCRIPTION OF THE INVENTION

Polymorphisms in the region of the interleukin (IL)28B gene on chromosome 19 have been associated with pegylated-interferon (PEG-IFN) and ribavirin treatment response mainly in genotype 1 HCV infections. However there are few data in HCV genotype 4 (HCV-4) infection. The inventors evaluated, in a unique well-characterized cohort of HCV-4 chronic hepatitis patients, the association of IL28B polymorphism with response to treatment or liver disease severity. This study included 164 HCV-4 patients from different ethnic groups (Egyptian, European and Sub-Saharan African). Among these patients, 82 were studied for response and 160 for disease severity. Free DNA extracted from all these 164 patient serum samples were analyzed by direct sequencing of the SNP rs12979860 of IL28B. Genetic and bio-clinical features from patients having sustained virological response (43 SVR patients) and from those who did not respond to treatment or had a relapse after the end of the treatment (39 NR patients) were compared. IL28B polymorphism was compared between the 78 patients with mild fibrosis (METAVIR score F0-F1) and the 82 with advanced fibrosis (F2-F4). Our data showed that IL28B rs12979860 CC genotype was associated with a better treatment response rate (p-value=0.0008). The response rates were 81.8%, 46.5% and 29.4% for genotype CC, CT and TT respectively. No significant relationship was found between rs12979860 and the severity of the disease (p-value=0.37). The SNP rs12979860 is strongly associated with SVR in patients infected with HCV-4, but not with liver disease severity. Analysis of IL28B genotype might be used to guide treatment for these patients.

Accordingly, the present invention relates to a method for testing whether a patient infected with HCV genotype 4 could achieve a sustained virological response (SVR) to the combination of interferon-alpha and ribavirin comprising determining the patient's genotype for the single nucleotide polymorphism rs12979860 wherein the presence of the better response allele C indicates a high chance to achieve a sustained virological response (SVR), and the presence of the allele T indicates a lower chance to achieve a sustained virological response (SVR).

Sustained viral response” or “SVR” means the undetectable serum HCV RNA at the conclusion of antiviral therapy and at a maximum of 24 weeks following the end of antiviral therapy. In some embodiments, SVR is measured at 12 weeks following the end of antiviral therapy (Martinot-Peignoux M, et al. Twelve weeks posttreatment follow-up is as relevant as 24 weeks to determine the sustained virologic response in patients with hepatitis C virus receiving pegylated interferon and ribavirin. Hepatology. 2010; 51(4):1122-6). SVR is also described by Dr. Steven L. Flamm in the Journal of the American Medical Association, Vol. 289, No. 18, pp. 2413 to 2417 (2003).

The term “interferon-alpha” or “IFN-alpha” as used herein refers to a family of related polypeptides that inhibit viral replication and cellular proliferation and modulate immune response. The term “IFN-alpha” includes naturally occurring IFN-alpha; synthetic IFN-alpha; derivatized IFN-alpha (e.g., PEGylated IFN-alpha, glycosylated IFN-alpha, and the like); and analogs of naturally occurring or synthetic IFN-alpha; essentially any IFN-alpha that has antiviral properties, as described for naturally occurring IFN-alpha. Suitable interferons alpha include, but are not limited to, naturally-occurring IFN-alpha (including, but not limited to, naturally occurring IFN-alpha2a, IFN-alpha2b) or recombinant interferon alpha. The term “IFN-alpha” also encompasses derivatives of IFN-alpha that are derivatized (e.g., are chemically modified) to alter certain properties such as serum half-life. As such, the term “IFN-alpha” includes glycosylated IFN-alpha; IFN-alpha derivatized with polyethylene glycol (“PEGylated IFN-alpha” or “PEG-IFN”); and the like. PEGylated IFN-alpha, and methods for making same, is discussed in, e.g., U.S. Pat. Nos. 5,382,657; 5,981,709; and 5,951,974. PEGylated IFN-alpha encompasses conjugates of PEG and any of the above-described IFN-alpha molecules, including, but not limited to, PEG conjugated to interferon alpha-2a (Roferon, Hoffman La-Roche, Nutley, N.J.), interferon alpha 2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferons alpha (Infergen (InterMune, Inc., Brisbane, Calif.).

The term “ribavirin” denotes the 1-β-D-ribofuranosyl-1,2,4-triazole-3-carboxamide compound. Ribavirin is a nucleoside analog available from ICN Pharmaceuticals, Inc., Costa Mesa, Calif., and is described in the Merck Index, compound No. 8199, Eleventh Edition. Its manufacture and formulation is described in U.S. Pat. No. 4,211,771.

As used herein, the term “polymorphism” or “allelic variant” means a mutation in the normal sequence of a gene. Allelic variants can be found in the exons, introns, or the coding region of the gene, or in the sequences that control expression of the gene. The term “Single nucleotide polymorphism” or “SNP” means a single nucleotide variation in a genetic sequence that occurs at appreciable frequency in the human population.

As used herein, the SNP that is concerned by the invention is described as follows:

| | dbSNP sequence | Allele 1 | Allele 2 |
|---|---|---|---|
| rs ID | (5′->3′) | (dbSNP) | (dbSNP) |
| rs12979860 | CTGAACCAGGGAGCTCCCC | C | T |
| | GAAGGCG[C/T]GAACCAG | | |
| | GGTTGAATTGCACTCCGC | | |
| | (SEQ ID NO: 1) | | |

According to the invention the allele that is associated with a high chance to achieve a SVR is C. Said allele is defined in the present invention as the “better response allele”.

According to the invention the allele that is associated whit a lower chance to achieve a SVR is T. Said allele is defined in the present invention as the “worse response allele”.

In another embodiment of the invention, the sample obtained from the patient comprises bodily fluids (such as blood, saliva or any other bodily secretion or derivative thereof), and/or tissue extracts such as homogenates or solubilized tissue obtained from the patient. In a preferred embodiment, the sample to be tested is blood.

According to the invention, the sample comprises nucleic acids, wherein nucleic acids may be genomic DNA, heterogenous nuclear RNA (hnRNA, also referred as incompletely processed single strand of ribonucleic acid) and/or cDNA.

According to the invention, the determination of the said genotype may be determined by nucleic acid sequencing, PCR analysis or any genotyping method known in the art. Examples of such methods include, but are not limited to, chemical assays such as allele specific hybridization, primer extension, allele specific oligonucleotide ligation, sequencing, enzymatic cleavage, flap endonuclease discrimination; and detection methods such as fluorescence, chemiluminescence, and mass spectrometry.

For example, the presence or absence of said polymorphism may be detected in a RNA or DNA sample, preferably after amplification. For instance, the isolated RNA may be patiented to couple reverse transcription and amplification, such as reverse transcription and amplification by polymerase chain reaction (RT-PCR), using specific oligonucleotide primers that are specific for the polymorphism or that enable amplification of a region containing the polymorphism. According to a first alternative, conditions for primer annealing may be chosen to ensure specific reverse transcription (where appropriate) and amplification; so that the appearance of an amplification product be a diagnostic of the presence of the polymorphism according to the invention. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to genotype the polymorphism (or identify the allele).

Actually numerous strategies for genotype analysis are available (Antonarakis et al., 1989; Cooper et al., 1991; Grompe, 1993). Briefly, the nucleic acid molecule may be tested for the presence or absence of a restriction site. When a base polymorphism creates or abolishes the recognition site of a restriction enzyme, this allows a simple direct PCR genotype the polymorphism. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a patient is first patiented to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base polymorphisms. According to this method, two oligonucleotides are constructed that hybridize to adjacent sequences in the target nucleic acid, with the join sited at the position of the polymorphism. DNA ligase will covalently join the two oligonucleotides only if they are perfectly hybridized to one of the allele.

Therefore, useful nucleic acid molecules, in particular oligonucleotide probes or primers, according to the present invention include those which specifically hybridize the one of the allele of the polymorphism.

Oligonucleotide probes or primers may contain at least 10, 15, 20 or 30 nucleotides. Their length may be shorter than 400, 300, 200 or 100 nucleotides.

The invention also contemplates a method for testing whether a patient infected with HCV genotype 4 could achieve a sustained virological response (SVR) to the combination of interferon-alpha and ribavirin, or will achieve a non response to said combination, by determining the genotype of at least one single nucleotide polymorphism in linkage disequilibrium (LD) with rs12979860.

The term “linkage disequilibrium” (LD) refers to a population association among alleles at two or more loci. It is a measure of co-segregation of alleles in a population. Linkage disequilibrium or allelic association is the preferential association of a particular allele or any other genetic marker with a specific allele, or genetic marker at a nearby chromosomal location more frequently than expected by chance for any particular allele frequency in the population. Accordingly, two particular alleles at different loci on the same chromosome are said to be in LD if the presence of one of the alleles at one locus tends to predict the presence of the other allele at the other locus.

Linked variants are readily identified by determining the degree of linkage disequilibrium (LD) between the allele genotyped for rs12979860 and a candidate linked allele at a polymorphic site located in the chromosomal region 19q13.13 or elsewhere on chromosome 19. The candidate linked variant may be an allele of a polymorphism that is currently known. Other candidate linked variants may be readily identified by the skilled artisan using any technique well-known in the art for discovering polymorphisms. One of the most frequently used measures of linkage disequilibrium is r, which is calculated using the formula described by Devlin et al. (Genomics, 29(2):311-22 (1995)). “r” is the measure of how well an allele X at a first locus predicts the occurrence of an allele Y at a second locus on the same chromosome. The measure only reaches 1.0 when the prediction is perfect (e.g. X if and only if Y). Typically, SNPs considered in LD with rs12979860 according to the invention present a r2 value greater than 0.2, preferably greater than 0.4 and even more preferably greater than 0.5.

In some embodiments, the method of the invention is performed by a laboratory that will generate a test report. The test report will thus indicates whether the better response allele is present or absent for the genotypes polymorphism, and preferably indicates whether the patient is heterozygous or homozygous for the better response allele. The test report can also indicates whether the worse response allele is present or absent for the genotypes polymorphism, and preferably indicates whether the patient is heterozygous or homozygous for the worse response allele. Accordingly, if the patient is homozygous for the better response allele, then the test report further indicates that the patient is positive for a genetic marker associated with a likely response to the treatment, while if the individual is heterozygous for the better response allele or is homozygous for the worse response allele, then the test report further indicates that the patient is negative for a genetic marker associated with a likely response to the treatment. In some embodiments, the test result will include a probability score for achieving a beneficial response to the treatment, which is derived from running a model that various patient parameters (e.g., age, gender, weight, race, test results for other pharmacogenetic markers for the treatment) and disease parameters (e.g., baseline viral load, and degree of fibrosis) that are associated with treatment response in the relevant disease population. The weight given to each parameter is based on its contribution relative to the other parameters in explaining the inter-individual variability of response to the treatment in the relevant disease population. This response probability score may be used as a guide in selecting a therapy or treatment regimen for the patient. In some embodiments, the test report may be thus generated by a computer program for establishing such a score.

In some embodiments, the patient would be tested prior to initiation of the treatment, but it is envisioned that such testing could be performed at any time after the individual is administered with the treatment. For example, the treating physician may be concerned that the patient has not responded adequately and desires to test the individual to determine whether continued treatment is warranted.

A further object of the invention is a kit for performing the methods of the invention, comprising at least one primer and/or at least one probe for amplification of a sequence comprising the polymorphisms of the invention and instructions for use.

In one embodiment of the invention, the primer or probe may be labelled with a suitable marker. In another embodiment of the invention, the primer or probe may be coated on an array.

The kit can include clinical data such as phenotype of the macula and questionnaire of the patient.

A further object of the invention relates to a method for the treatment of an HCV genotype 4 infection.

In the context of the invention, the term “treating” or “treatment”, as used herein, means reversing, alleviating, inhibiting the progress of, or preventing the disorder or condition to which such term applies, or one or more symptoms of such disorder or condition.

In a particular embodiment, the said method comprises the following steps

a) testing whether the patient infected with HCV genotype 4 could achieve a sustained virological response (SVR) to the combination of interferon-alpha and ribavirin by performing the method of the invention; and

b) administering a combination of interferon-alpha and ribavirin to said patient, if the said patient has been considered as positive for a genetic marker associated with a likely response to said combination, at step a) above.

In case where the patient has been considered as negative for a genetic marker with a likely response to said combination, and if the disease is mild, the treatment might be delayed.

The method of the invention allows to define a subgroup of patients who will be responsive (“responder”) or not (“non responder”) to the treatment with interferon-alpha and ribavirin.

Typically, if the patient has been considered as positive for a genetic marker associated with a likely response to said combination, the duration of treatment may be shortened.

A further object of the invention is then the use of interferon-alpha and ribavirin, for the preparation of a medicament for treating a patient infected with HCV genotype 4, which patient being classified as responder by the method as above described.

The invention will be further illustrated by the following figures and examples. However, these examples and figures should not be interpreted in any way as limiting the scope of the present invention.

### FIGURES

FIG. 1: IL28B polymorphism according to sustained virological response (SVR): IL28B rs12979860 CC genotype was associated with a better treatment response rate. The response rates were 81.8%, 46.5% and 29.4% for genotype CC, CT and TT respectively.

FIG. 2: IL28B genotype distribution according to ethnicity people. The genotype distributions for SNP rs12979860 were significantly different between the three ethnic groups: Egyptian (A), European (B) and Sub-Saharan African (C).

FIG. 3: IL28B polymorphism according to Fibrosis stage (METAVIR score). IL28B is not associated with Fibrosis stage (METAVIR score).

### EXAMPLE

### Material & Methods

Patient and Samples

One-hundred and sixty four patients with an established diagnosis of HCV-4 chronic hepatitis with detectable anti-HCV antibodies, and detectable serum HCV RNA were included in this study. A percutaneous liver biopsy was performed in 160 patients and a METAVIR score was allocated (16).

Eighty-two patients were included in the response cohort if they meet the following criteria:
An established diagnosis of chronic hepatitis C with detectable anti-HCV antibodies, and detectable serum HCV RNA.
Receiving the same complete treatment of either PEG-IFN-2b (Viraferonpeg, Schering Plough Corporation, Kenilworth, N.J.) at a dose of 1.5 μg/kg/week and ribavirin (Rebetol, Schering Plough Corporation Kenilworth, N.J.) at a dose of 800-1,200 mg/kg/day, or PEG-IFN-2a at a dose of 180 μg/week (Pegasys, Roche Corporation, Kenilworth, N.J.) and weight-based ribavirin 1,000-1,200 mg/kg/day (Copegus, Roche). Duration of treatment was 48 weeks.
Adequate follow-up: detection of serum HCV RNA by RT-PCR was performed at the end of treatment and 6 months after the end of treatment.

All the patients received an informed consent for the collection and storage of serum sample and liver biopsy, for testing of their DNA for research purposes consistent with the current study. The study was approved by a central ethics committee and was conducted in accordance with the provisions of the declaration of Helsinki and Good Clinical Practice guidelines.

HCV Viral Load Testing, HCV Genotyping

Serum HCV-RNA was retrospectively quantified by the VERSANT® HCV-RNA 3.0 (bDNA) Assay (Siemens Medical Solutions, Puteaux, France) with a quantification range of 615-7 690 000 IU/ml. Serum samples below 615 IU/ml were evaluated with the VERSANT® HCVRNA Qualitative Assay (HCV Qual (TMA), Siemens Medical Solutions, Puteaux, France) with a detection limit of 9.6 IU/ml. HCV genotyping was performed by reverse hybridization (InnoLIPA HCV; Innogenetics, Gent, Belgium) in all patients.

DNA Extraction and IL-28B Genotyping

The genomic region associated with HCV treatment response lies on chromosome 19 and contains multiple SNPs in linkage disequilibrium around the IL28B gene. The SNP rs12979860, which is located 3-kb upstream of the IL28B gene and displayed the highest association signal for SVR was selected for this study (8). Primers used are available on request. One hundred sixty four patients were genotyped for rs12979860 using direct sequencing (AmpliTaq Gold® DNA polymerase and BigDye® terminator v1.1 cycle sequencing kit, Applied Biosystems, Warrington, United Kingdom). Free circulating DNA was extracted from 500 μl serum samples (QIAamp Circulating Nucleic Acid Kit; Qiagen Inc, Valencia, Calif., USA). The PCR product were separated on an ABI3130 sequencer, and analysed with SEQSCAPE® 2.6 (Applied Biosystems, Warrington, United Kingdom).

Statistical Analysis

In this work, the genetic and bio-clinical features have been compared between patients having SVR (43 responder patients) and those who did not respond to PEG-IFN plus ribavirin treatment or had a relapse after the end of the treatment (39 non-responder patients). We evaluated the statistical significance of the relationships between bio-clinical characteristics (age, gender, fibrosis (METAVIR score F0-1 vs F2-4), Viral load at treatment given as log 10 international units, Body Mass Index, ethnic origin (Egyptian, European, Sub-Saharan African) and the response phenotype (responder/non-responder) by using chi-square test for discrete variables and Student's t-test for continuous variables. For multivariate analyses, we consider multivariate logistic regression model (17).

The test for association between the IL28B polymorphism (rs12979860) and the binary phenotype (responder/non-responder) was carried out using the Cochran-Armitage trend test (18). For all these tests, statistical significance was considered as p-values less than 0.05. All these analyses were carried out using the R software package (http://cran.rproject.org/index.html).

Results:

Patients:

A total of 164 HCV-4 infected patients, 43 (26%) women and 121 (74%) men, were studied. Baseline characteristics of these 164 patients are presented in Table 1. The median age at onset of therapy was 44 years old [range: 22-66]. This study recruited patients from three different ethnic groups (as self-reported by the patient) with 70 (43%) Egyptian, 53 (32%) European and 37 (23%) Sub-Saharan African. A liver biopsy was performed in 160 of them who were included in a severity analysis. Seventy eight (49%) patients have a mild fibrosis (F0 or F1 with METAVIR score) and 82 (51%) have a moderate or severe fibrosis (F2, F3 or F4). Moreover, 82 patients, who receive 48 weeks of SOC, were included in a response cohort. Among these patients, 43 patients (52%) achieved a SVR and 39 failed to treatment (28 (32%) developed a non response and 11 (16%) were relapsers).

The RVR defined by an undetectable HCV RNA at week 4 after treatment initiation, was available for 59 patients. Among these 59 patients, HCV RNA was undetectable in 15 patients (25.4%).

Relationship Between IL28B rs12979860 Polymorphism and Treatment Response (Univariate Analysis):

Among our 82 treated patients the proportion of rs12979860 CC was 26.8%; CT was 52.4% and TT was 20.8%. Univariate analysis showed no relationship with response type and either age at therapy (p-value=0.83), gender (p-value=0.81), ethnicity (p-value=0.98), fibrosis (pvalue=0.21), Body Mass Index (p-value=0.52) However a significant relationship was observed between response type and baseline viral load (p-value=0.01). In our study, responder patients had a lower mean baseline viral load than non-responder patients. No significant difference was found between mean baseline viral load values among the three ethnic groups (p-values=0.53). Moreover, no significant relationship between mean baseline viral load values and IL28B rs12979860 genotypes was shown (p-value=0.28).

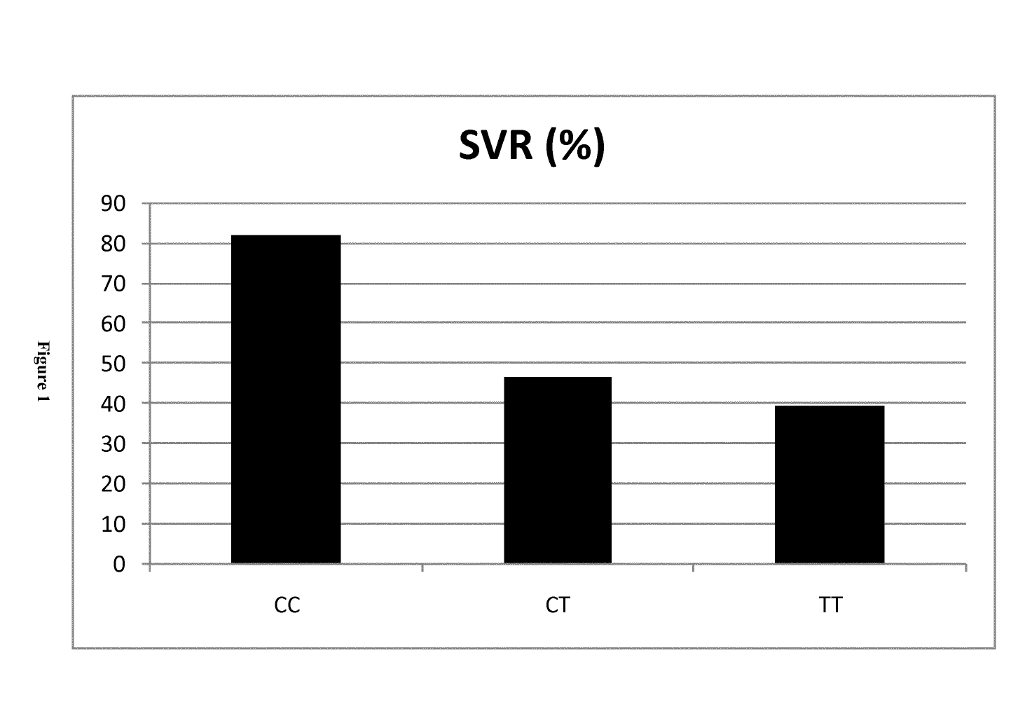

The genotype distributions for IL28B polymorphism (rs12979860) were significantly different between responder and non-responder patients (trend test: p-value=0.0008). In our series, the odds ratio of being a responder for genotype CC genotype as compared to genotype CT and TT was 6.3 [95% CI: 1.83-21.6]. The response rates were 81.8% [65.7-97.9], 46.5% [31.6-61.4] and 29.4% [7.7-51.1] for genotype CC, CT and TT respectively. SVR according to IL28B genotypes are presented in FIG. 1. No significant deviation from Hardy-Weinberg equilibrium was observed for SNP rs12979860 (p-value=0.67).

Among these treated patients, the genotype distributions for SNP rs12979860 were significantly different between the three ethnic groups (frequencies of the C allele were 60.7%, 51.8% and 27.3% for patients of Egyptian, European and Sub-Saharan Africa origin respectively).

It is worth noting that in the 59 treated patients whose HCV RNA was available at week 4, there was a significantly higher rate of SVR in patients who achieved RVR (p-value=0.004). Among these 59 patients, 15 (25.4%) achieved RVR, of whom 13 (86.7%) obtained SVR.

Among those 44 patients who did not achieve RVR, 17 (38.6%) had SVR. Moreover, there was a borderline statistically significant relationship (trend test: p-value=0.05) between the genotype distributions for IL28B polymorphism and RVR. For the 15 patients with RVR, the IL28B genotype distribution were 7 (46.7%) CC, 6 (40.0%) CT and 2 (13.3%) TT. For the 44 patients without RVR, the IL28B genotype distribution were 8 (18.2%) CC, 25 (56.8%) CT and 11 TT (25.0%).

Relationship Between IL28B rs12979860 Polymorphism and Treatment Response (Multivariate Analysis):

A multivariate logistic regression analysis was performed including baseline viral load values at treatment (scale and centered values) and rs12979860 genotypes (additive/multiplicative genetic models) as the explanatory variables and responder/non-responder status as the dependent variable. The genetic and viral load variables showed a significant effect providing additional, non redundant information on the response phenotype (baseline viral load: pvalue=0.02, rs12979860: p-value=0.007). The inclusion of the ethnic group information did not modify the results.

Relationship Between IL28B rs12979860 Polymorphism and Fibrosis Stage:

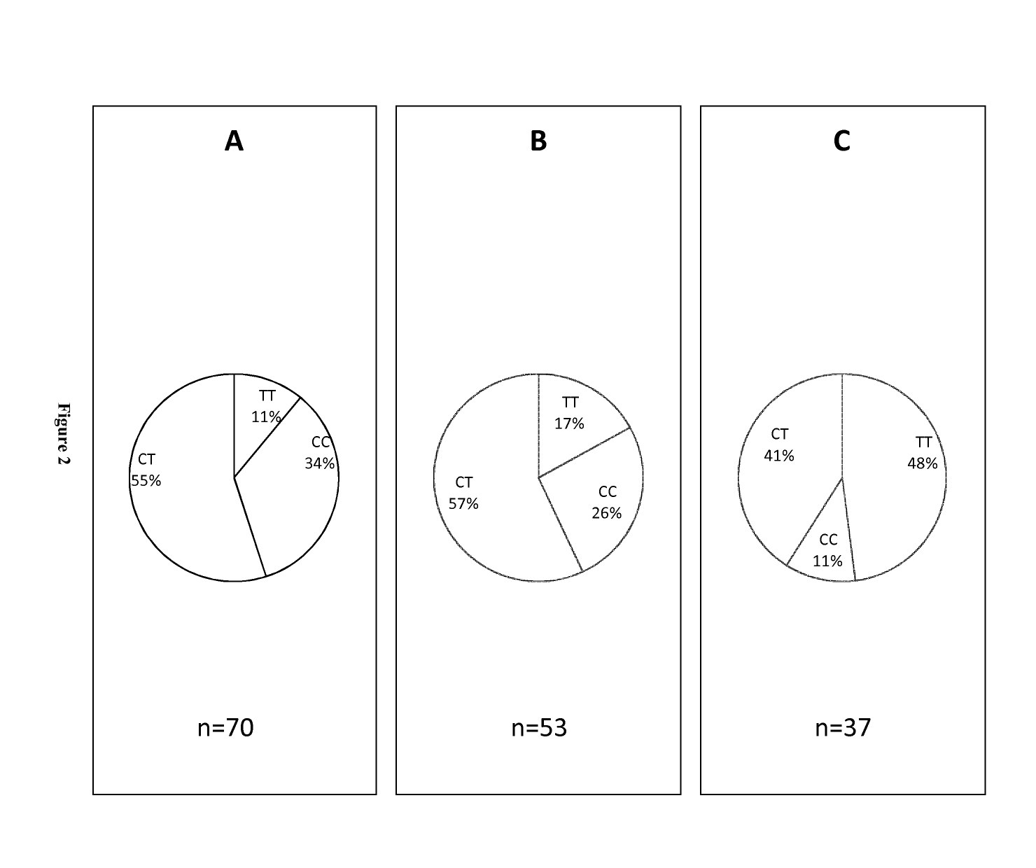

This is a large mono-centric cohort of 164 patients with HCV-4 infection, from three different ethnic groups with 70 (43%) Egyptian, 53 (32%) European and 37 (23%) Sub-Saharan African. A liver biopsy was performed in 160 of whom 78 (49%) patients had a mild fibrosis (F0-F1) and 82 (51%) a moderate to severe fibrosis (F2-F4). The genotype distributions for SNP rs12979860 were significantly different between the three ethnic groups (p-value<0.0002). Frequencies of the C allele were 61.4%, 54.7% and 31.0% for patients of Egyptian, European and Sub-Saharan Africa origin respectively. IL28B genotype distribution is presented according to ethnicity in FIG. 2.

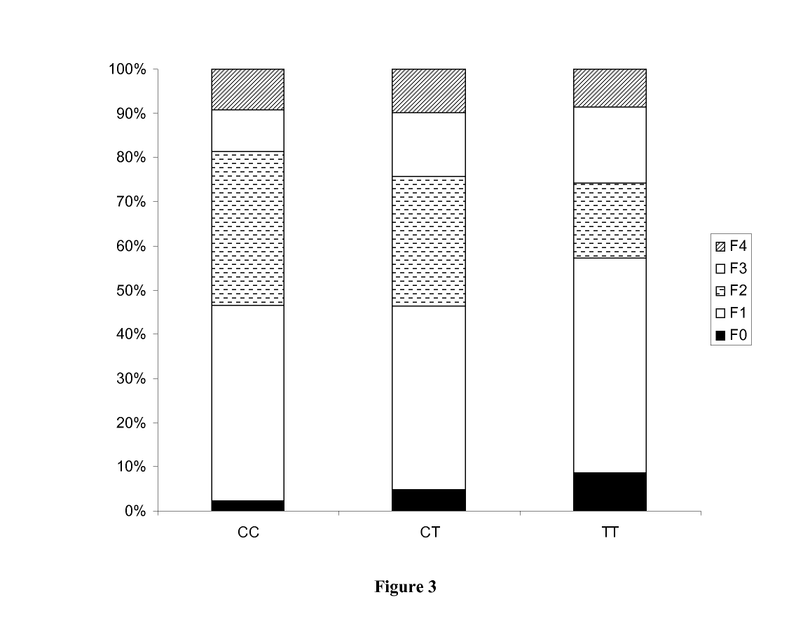

No significant relationship between IL28B rs12979860 and fibrosis stage was observed (ns; p-value=0.37). FIG. 3 represents fibrosis stage (METAVIR score) according to IL28B genotypes.

Discussion:

This unique cohort in HCV-4 infected populations has allowed us to analyze relationship between rs12979860 and treatment response or fibrosis stage, not evaluated previously.

This study was performed in a large unique mono-centric cohort of 164 patients with HCV-4 infection. In this cohort, three different ethnic groups were represented with 70 (43%) Egyptian, 53 (32%) European and 37 (23%) Sub-Saharan African. This unique ethnic population has not been yet evaluated for relationship between IL28B polymorphism and HCV infection patterns. A liver biopsy was performed in 160 patients of whom 78 (49%) had a mild fibrosis (F0-F1) and 82 (51%) a moderate to severe fibrosis (F2-F4). Eighty two patients received 48 weeks of SOC. Among these, 43 patients (52%) obtained a SVR and 39 failed to treatment (28 (32%) obtained a non response and 11 (16%) were relapsers).

Among our treated patients the proportion of rs12979860 CC was 26.8%; CT was 52.4% and TT was 20.8%. Interestingly, in a large cohort from Dukes University, the rs12979860 CC was most common in genotype 3 patients (55%), followed by genotype 2 (46%) and then genotype 1 (33.5%) (19).

To the best of our knowledge, this is the first study that specifically examines the relationship between IL28B rs12979860 CC genotype, treatment response and liver severity in patients with chronic HCV-4 infections. We showed that the CC genotype is significantly associated with a better response rate for patients with chronic HCV-4 infection. We did not find a significant relationship between baseline viral load and the genotype but we cannot rule out the hypothesis of a loss of power due to the relatively small sample size. Interestingly, the inclusion of the ethnic group information did not modify the results.

Rapid clearance of HCV RNA (RVR) obtained in 15 among 59 patients was strong predictors of SVR, and was also associated with rs12979860 CC genotype.

Some previous studies identify genetic or molecular markers associated with fibrosis stage in chronic hepatitis C (20-21). When investigating the fibrosis stage, no significant relationship between IL28B rs12979860 and the severity of the disease was observed. Indeed, pathways associated with fibrosis progression or response to treatment must be different.

The IFN-λ proteins, encoded by the IL28A/B and IL29 genes, have antiviral properties (22-23). Although all of the identified variants associated with response to treatment of HCV chronic infection in previous studies lie in or near the IL28B gene, none of them has an obvious effect on the function of this gene (24). Of course, these new genetic predictive factors will have to compete with other predictors of response and will have to be validated in large prospective studies. The probability of SVR essentially depends on the viral genotype and viral load, but also on viral kinetic (RVR)(25). What will be the importance of this genetic predictor among all others?

In the near future, SOC treatment of HCV will include the addition of direct-acting antivirals (DAAs) with a protease inhibitor to PEG-IFN plus ribavirin, but only for HCV genotype 1 patients (26). Whereas, in genotype 4 patients, the SOC will remain PEG-IFN and ribavirin, thus IL28B polymorphism may remain an important associated factor with response. Further studies will be needed to demonstrate if genotype 4 infected patients with good predictors of response, including IL28B CC, may benefit from shorten therapy.

TABLE 1
Characteristics of 164 patients with chronic hepatitis C

| Variable | Patients | |
|---|---|---|
| N | 164 | |
| Sex: male (%)/female (%) | 43 (26.2)/121 (73.8) | |
| Age (years) ^{a} | 44.3 ± 9.3 (22-66) | |
| Ethnic group [n(%)] | | |
| Egyptian | 70 | (42.7) |
| Caucasian | 53 | (32.3) |
| Black african | 37 | (22.6) |
| Others | 3 | (1.8) |
| Unknown | 1 | (0.6) |
| Alanine aminotransferase (ALT) IU/L ^{a} | 103.0 ± 70.8 (18-397) | |
| HCV genotype 4 subtypes [(n (%)] | | |
| Indeterminate | 64 | (39) |
| a | 29 | (17.7) |
| acd | 29 | (17.7) |
| c | 1 | (0.6) |
| cd | 7 | (4.3) |
| d | 4 | (2.4) |
| e | 4 | (2.4) |
| f | 6 | (3.7) |
| g | 1 | (0.6) |
| h | 19 | (11.6) |
| Fibrosis stage [n (%)] | | |
| 0 | 8 | (4.9) |
| 1 | 70 | (42.7) |
| 2 | 45 | (27.4) |
| 3 | 22 | (13.4) |
| 4 | 15 | (9.1) |
| Unknown | 4 | (2.5) |
| IL-28B genotypes frequency [(n (%)] | | |
| CC | 43 | (26.2) |
| CT | 85 | (51.8) |
| TT | 36 | (22) |
| Treatment [(n (%)] | | |
| SVR | 43 | (26.2) |
| RR | 11 | (6.7) |
| NR | 28 | (17.1) |
| Untreated | 82 | (50) |

^{a} Results are expressed as mean ± SD (range).
NRs, non-responders; SVRs, sustained virological responders; RRs, responder-relapser patients

### REFERENCES

Throughout this application, various references describe the state of the art to which this invention pertains. The disclosures of these references are hereby incorporated by reference into the present disclosure.
1—Antaki N, Craxi A, Kamal S, et al. The neglected hepatitis C virus genotypes 4, 5 and 6: an international consensus report. Liver Int. 2010 March; 30(3):342-55.
2—Kamal S M. Hepatitis C virus genotype 4 therapy: progress and challenges. Liver Int. 2011; 31 Suppl 1:45-52.
3—Manns M P, McHutchison J G, Gordon S C, et al. Peginterferon alfa-2b plus ribavirin compared with interferon alfa-2b plus ribavirin for initial treatment of chronic hepatitis C: a randomised trial. Lancet 2001; 358: 958-65.
4—Fried M W, Shiffman M L, Reddy K R, et al. Peginterferon alfa-2a plus ribavirin for chronic hepatitis C virus infection. N Engl J Med 2002; 347: 975-82.
5—Bronowicki J P, Ouzan D, Asselah T, et al. Effect of ribavirin in genotype 1 patients with hepatitis C responding to pegylated interferon alfa-2a plus ribavirin. Gastroenterology 2006; 131: 1040-8.
6—Asselah T, Bieche I, Sabbagh A, et al. Gene expression and hepatitis C virus infection. Gut 2009; 58:846-858.
7—Asselah T, Estrabaud E, Bieche I, et al. Hepatitis C: viral and host factors associated with non-response to pegylated interferon plus ribavirin. Liver Int. 2010; 30(9):1259-69.
8—Ge D, Fellay J, Thompson A J et al. Genetic variation in IL28B predicts hepatitis C treatment-induced viral clearance. Nature 2009; 461:399-401.
9—Suppiah V, Moldovan M, Ahlenstiel G et al. IL28B is associated with response to chronic hepatitis C interferon-alpha and ribavirin therapy. Nat Genet 2009; 41:1100-1104.
10—Tanaka Y, Nishida N, Sugiyama M et al. Genome-wide association of IL28B with response to pegylated interferon-alpha and ribavirin therapy for chronic hepatitis C. Nat Genet 2009; 41:1105-1109.
11—Rauch A, Kutalik Z, Descombes P et al. Genetic variation in IL28B is associated with chronic hepatitis C and treatment failure: a genome-wide association study. Gastroenterology. 2010; 138(4):1338-45, 1345.e1-7.
12—Moghaddam A, Melum E, Reinton N et al. IL28B genetic variation and treatment response in patients with hepatitis C virus genotype 3 infection. Hepatology. 2011; 53(3):746-54.
13—Mangia A, Thompson A J, Santoro R et al. An IL28B polymorphism determines treatment response of hepatitis C virus genotype 2 or 3 patients who do not achieve a rapid virologic response. Gastroenterology. 2010; 139(3):821-7, 827.e1.
14—Yu M L, Huang C F, Huang J F et al. Role of interleukin-28B polymorphisms in the treatment of hepatitis C virus genotype 2 infection in Asian patients. Hepatology. 2011; 53(1):7-13.
15—Mahmoud A. Khattab, Peter Ferenci, Stephanos J. Hadziyannis, et al. Management of hepatitis C virus genotype 4: Recommendations of An International Expert Panel Journal of Hepatology, Volume 54, Issue 6, June 2011, Pages 1250-1262
16—Bedossa P, Poynard T. An algorithm for the grading of activity in chronic hepatitis C. The METAVIR Cooperative Study Group. Hepatology. 1996; 24(2):289-93.
17—Gerald van Belle, Lloyd D. Fisher, Patrick J. Heagerty et al. Lumley. Biostatistics: A Methodology for the Health Sciences, 2nd Edition, 2004, Wiley, John & Sons
18—Sasieni, P D (1997). From genotypes to genes: doubling the sample size. Biometrics 53, 1253-1261.
19 McCarthy J J, Li J H, Thompson A et al. Replicated association between an IL28B gene variant and a sustained response to pegylated interferon and ribavirin. Gastroenterology. 2010 June; 138:2307-14.
20—Marcolongo M, Young B, Dal Pero F, et al. A seven-gene signature (cirrhosis risk score) predicts liver fibrosis progression in patients with initially mild chronic hepatitis C. Hepatology. 2009; 50(4):1038-44.
21—Asselah T, Bièche I, Laurendeau I, et al. Liver gene expression signature of mild fibrosis in patients with chronic hepatitis C. Gastroenterology. 2005; 129(6):2064-75.
22—Sheppard P, Kindsvogel W, Xu W et al. IL-28, IL-29 and their class II cytokine receptor IL-28R. Nat Immunol 2003; 4:63-68.
23—Marcello T, Grakoui A, Barba-Spaeth G et al. Interferons alpha and lambda inhibit hepatitis C virus replication with distinct signal transduction and gene regulation kinetics. Gastroenterology 2006; 131:1887-1898.
24—Asselah T. Genetic polymorphism and response to treatment in chronic hepatitis C: the future of personalized medicine. J Hepatol. 2010 March; 52(3):452-4.
25—Martinot-Peignoux M, Maylin S, Moucari R et al. Virological response at 4 weeks to predict outcome of hepatitis C treatment with pegylated interferon and ribavirin. Antivir Ther. 2009; 14(4):501-11.
26—Asselah T, Marcellin P. New direct-acting antivirals' combination for the treatment of chronic hepatitis C. Liver Int. 2011; 31 Suppl 1:68-77.

1. A method for testing whether a patient infected with HCV genotype 4 could achieve a sustained virological response (SVR) to the combination of interferon-alpha and ribavirin comprising
determining the patient's genotype for the single nucleotide polymorphism (SNP) rs12979860 by
i) isolating nucleic acid encoding the SNP rs12979860 from a sample from the subject;
ii) amplifying the isolated nucleic acid by contacting the isolated nucleic acid with oligonucleotide primers specific for amplifying the SNP rs12979860;
and
iii) analyzing a nucleotide sequence of amplification products generated in said step of amplifying;
wherein
if results obtained in said step of analyzing indicate that better response allele C is present at SNP rs12979860 of said nucleotide sequence, then concluding that the patient has a high chance to achieve a sustained virological response (SVR) and
if results obtained in said step of analyzing indicate that worse response allele T is present at SNP rs12979860 of said nucleotide sequence, then concluding that the patient has a lower chance to achieve a sustained virological response (SVR).

2. A method for testing whether a patient infected with HCV genotype 4 could achieve a sustained virological response (SVR) to the combination of interferon-alpha and ribavirin by determining the genotype of at least one single nucleotide polymorphism (SNP) in linkage disequilibrium (LD) with rs12979860 by
i) isolating nucleic acid encoding the at least one SNP
ii) amplifying the isolated nucleic acid by contacting the isolated nucleic acid with oligonucleotide primers specific for amplifying the at least one SNP
and
iii) analyzing a nucleotide sequence of amplification products generated in said step of amplifying; wherein
if results obtained in said step of analyzing indicate that the at least one SNP is present in said nucleotide sequence, then concluding that the patient has a high chance to achieve a sustained virological response (SVR), and
if results obtained in said step of analyzing indicate that the at least one SNP is not present in said nucleotide sequence, then concluding that the patient has a lower chance to achieve a sustained virological response (SVR).

3. A method for the treatment of an HCV genotype 4 infection in a patient in need thereof comprising
a) testing whether the patient infected with HCV genotype 4 could achieve a sustained virological response (SVR) to the combination of interferon-alpha and ribavirin by;
determining the patient's genotype for the single nucleotide polymorphism (SNP) rs12979860 by
i) isolating nucleic acid encoding the SNP rs12979860 from a sample from the subject;
ii) amplifying the isolated nucleic acid by contacting the isolated nucleic acid with oligonucleotide primers specific for amplifying the SNP rs12979860;
iii) analyzing a nucleotide sequence of amplification products generated in said step of amplifying; and if results obtained in said step of analyzing indicate that better response allele C is present at SNP rs12979860 of said nucleotide sequence, then concluding that the patient has a high chance to achieve a sustained virological response (SVR) and
b) administering a combination of interferon-alpha and ribavirin to the patient.
